(19) United States
(12) Patent Application Publication
(10) Pub. No.: US 2026/0335116 A1
(43) Pub. Date: Oct. 29, 2026

(54) MULTI-PIECE BREATHABLE WAIST PROTECTOR

(71) Applicant: CHI-SAN HUANG, Changhua City (TW)

(72) Inventor: CHI-SAN HUANG, Changhua City (TW)

(21) Appl. No.: 19/188,403

(22) Filed: Apr. 24, 2025

(51) Int. Cl. A61F 5/02

(52) U.S. Cl. CPC A61F 5/028

(57) ABSTRACT

A multi-piece breathable waist protector includes a first strap, a second strap, and two first protective sheets. The first strap is connected to one of the first protective sheets. The second strap is connected to the other first protective sheet. A second protective sheet is connected between the two first protective sheets through connecting members. The first protective sheets each have a middle portion protruding outward to form a first recessed space on an inner surface thereof. The first protective sheets each are formed with a plurality of first ventilation holes that are in communication with the first recessed space. The second protective sheet has a middle portion protruding outward to form a second recessed space on an inner surface thereof. The second protective sheet is formed with a plurality of second ventilation holes that are in communication with the second recessed space.

### TECHNICAL FIELD

The present invention relates to a waist protector, and more particularly to a multi-piece breathable waist protector for supporting and protecting the lumbar.

### BACKGROUND

With the development of the economy and science, the average life expectancy of people has reached a record high, and the emphasis on physical health has gradually increased to prevent the quality of life from being affected by physical discomfort or illness. Against this background, a waist protector, as a specially designed support device, aims to protect and support the lumbar. When in use, the waist protector is tightly attached to the user's waist, providing the necessary support and pressure to help maintain the correct posture of the lumbar vertebrae, and greatly reducing the risk of lumbar injury. For people with chronic low back pain or injuries, a waist protector can effectively alleviate discomfort, prevent further injury, and promote recovery.

When in use, a conventional waist protector surrounds and is secured on the user's waist through a Velcro strap, which makes it easy to adjust the position of the Velcro strap according to the user's waist size and adjust the tightness of the strap according to the user's preference. The waist protector is equipped with multiple pouches that contain metal plates to further enhance lumbar support.

However, when the strap is secured and tightened around the user's waist, the upper and lower ends of the metal plates will press against the user's body, restricting the user's motion range and preventing the body from bending freely. This design is not ergonomic and causes discomfort and inconvenience to the user. Besides, the conventional waist protector is not breathable enough, which makes it easy to generate a feeling of stuffiness when wearing the waist protector, thus affecting the overall comfort.

### SUMMARY

The primary object of the present invention is to provide a multi-piece breathable waist protector, comprising a first strap, a second strap, two first protective sheets, and a second protective sheet. The first strap has a first coupling portion on one side of an outer end thereof. The second strap has a second coupling portion on one side of an outer end thereof. The second coupling portion is configured for connecting the first coupling portion of the first strap. The two first protective sheets are made of a rigid material and connected to the inner ends of the first strap and the second strap, respectively. The first protective sheets are non-planar. The first protective sheets each have a middle portion protruding outward to form a first recessed space on an inner surface thereof. The first protective sheets each are formed with a plurality of first ventilation holes. The first ventilation holes are in communication with the first recessed space. When a user wears the multi-piece breathable waist protector, through the first recessed space, only two sides of the first protective sheet are in contact with the user's body.

The second protective sheet is made of a rigid material and located between the two first protective sheets and connected to each of the two first protective sheets through at least one connecting member. The second protective sheet is non-planar. The second protective sheet has a middle portion protruding outward to form a second recessed space on an inner surface thereof. The second protective sheet is formed with a plurality of second ventilation holes. The second ventilation holes are in communication with the second recessed space. When the user wears the multi-piece breathable waist protector, through the second recessed space, only two sides of the second protective sheet are in contact with the user's body.

In the present invention, the first protective sheet and the second protective sheet are made of a rigid material. The middle portion of the first protective sheet protrudes outward to form the first recessed space on the inner surface of the first protective sheet. The first protective sheet is formed with the plurality of first ventilation holes that are in communication with the first recessed space. The middle portion of the second protective sheet protrudes outward to form the second recessed space on the inner surface of the second protective sheet. The second protective sheet is formed with the plurality of second ventilation holes that are in communication with the second recessed space. Through the first recessed space and the second recessed space, the middle portions of the first protective sheet and the second protective sheet are not in contact with the user's body when in use. The first recessed space and the second recessed space are ventilated with the external air through the first ventilation holes and the second ventilation holes, thereby improving the overall air permeability, effectively reducing the stuffy feeling when the user wears the waist protector, and thus improving the wearing comfort.

In the present invention, the middle portion of the second protective sheet protrudes outward to form the second recessed space on the inner surface of the second protective sheet. When in use, only two sides of the second protective sheet are in contact with the user's body, and the middle portion of the second protective sheet is not in contact with the user's body and lumbar, thereby reducing the pressure on the user's lumbar. This is ergonomically designed to avoid discomfort and inconvenience.

### BRIEF DESCRIPTION OF THE DRAWINGS

FIG. 1 is a perspective view of the present invention;

FIG. 2 is an exploded view of the present invention;

FIG. 3 is a planar view of the present invention;

FIG. 4 is a sectional view taken along line A-A of FIG. 3;

FIG. 5 is a sectional view taken along line B-B of FIG. 3;

FIG. 6 is a top planar view of the first protective sheet of the present invention;

FIG. 7 is a top planar view of the second protective sheet of the present invention;

FIG. 8 is a first schematic view of the present invention when in use;

FIG. 9 is a second schematic view of the present invention when in use;

FIG. 10 is a schematic view of the present invention when in use, wherein the first protective sheet is in contact with the user's body; and

FIG. 11 is a schematic view of the present invention when in use, wherein the second protective sheet is in contact with the user's body.

### DETAILED DESCRIPTION

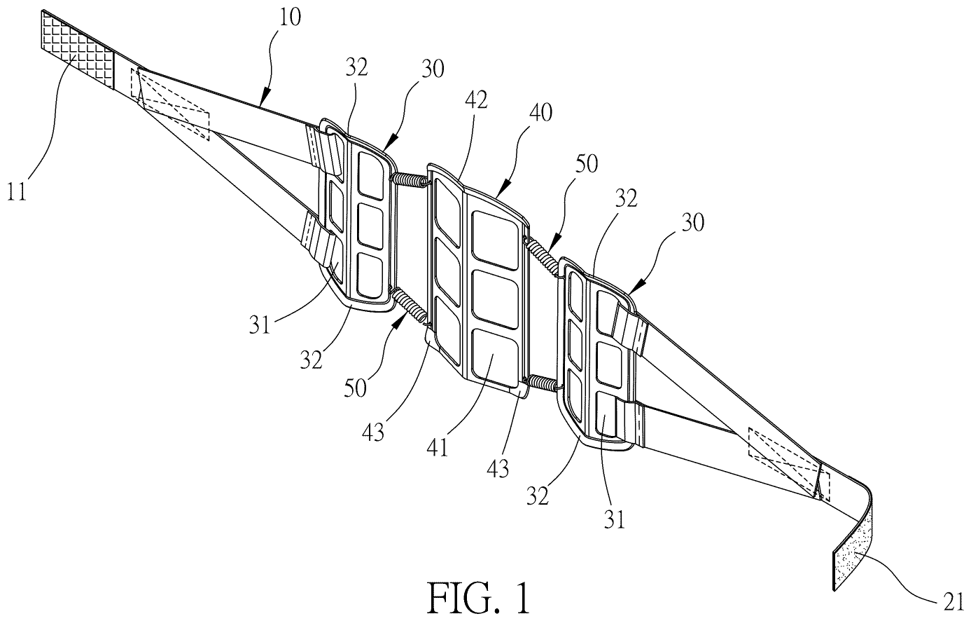

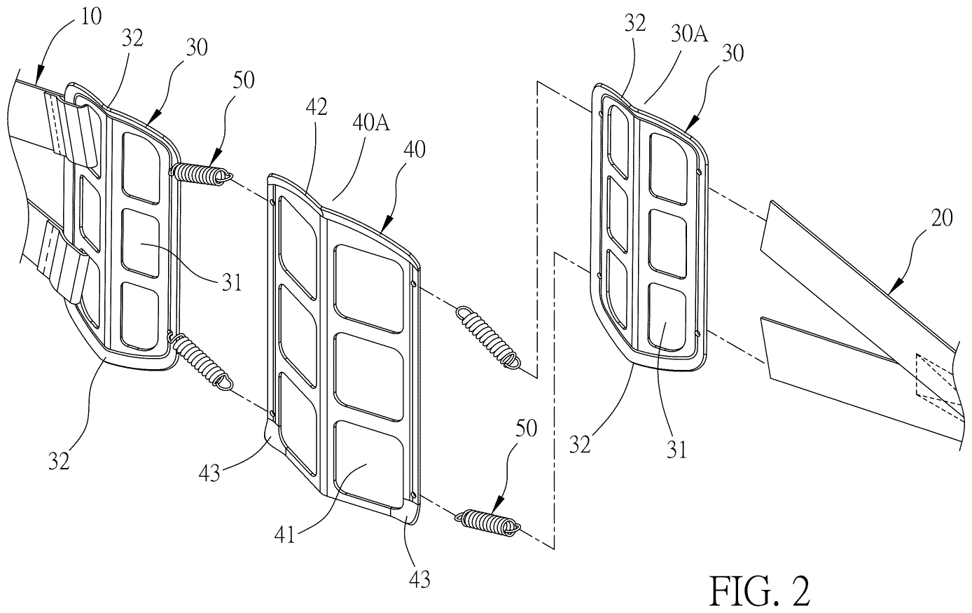

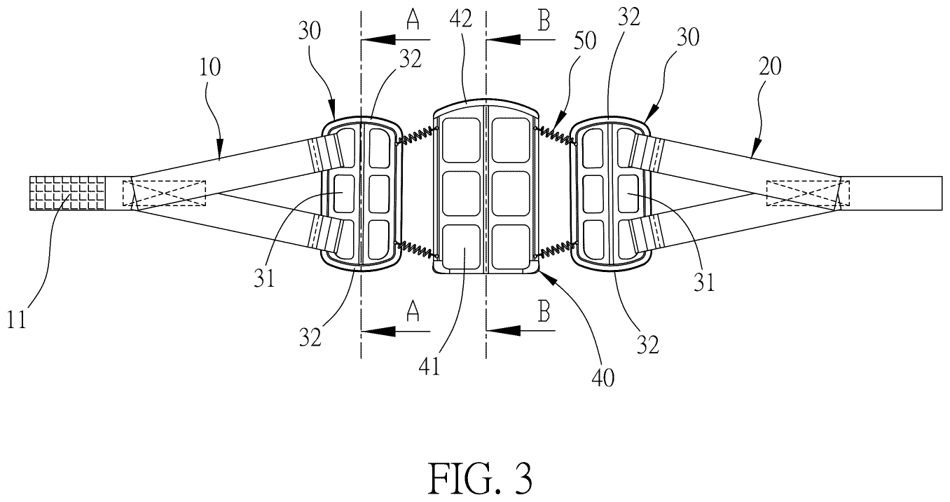

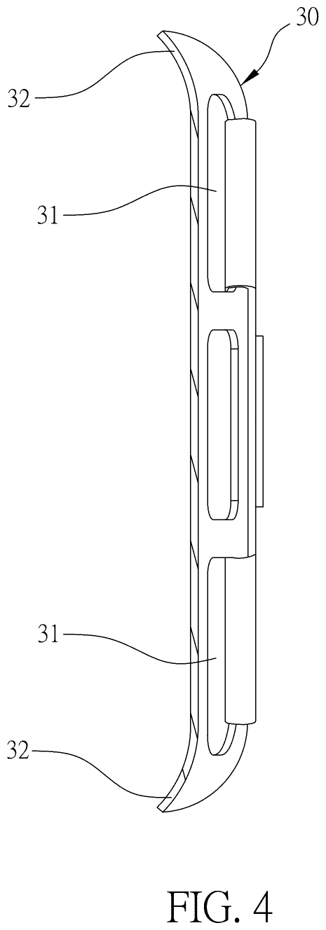

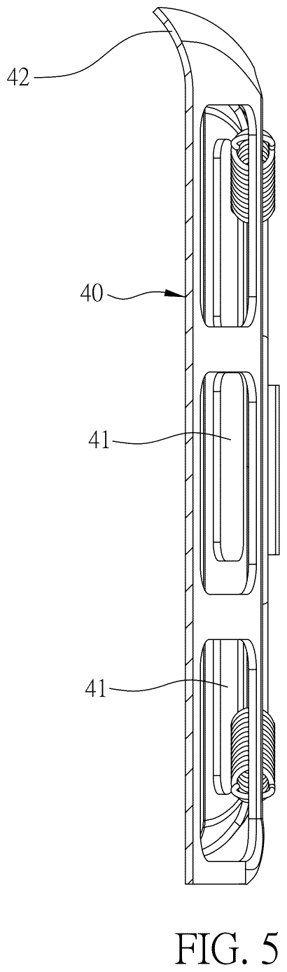

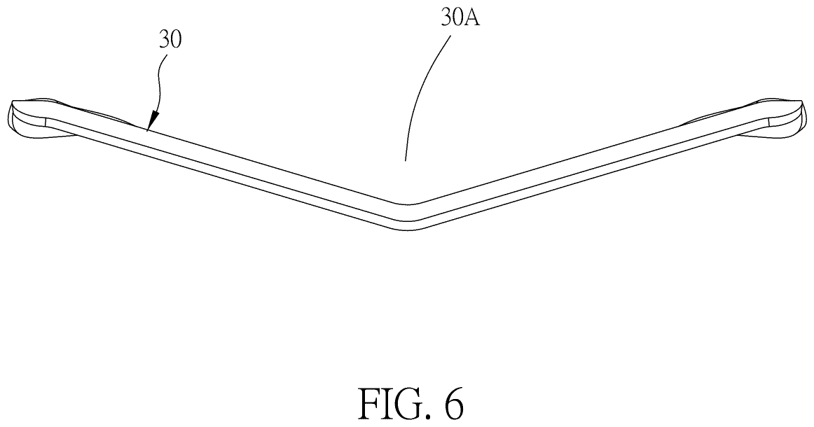

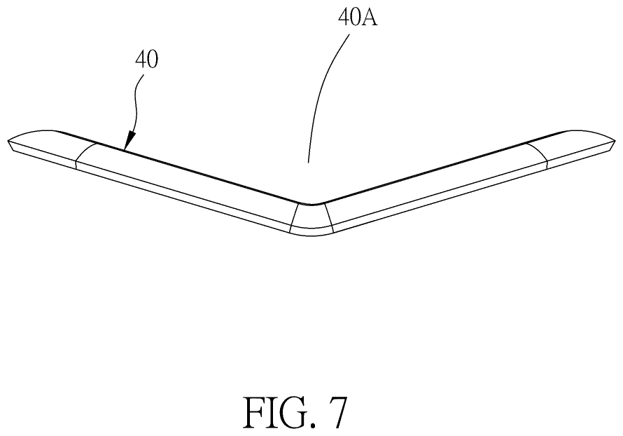

With reference to FIGS. 1-7, a multi-piece breathable waist protector according to a preferred embodiment of the present invention comprises a first strap 10, a second strap 20, two first protective sheets 30, and a second protective sheet 40.

The first strap 10 is made of fabric and has a first coupling portion 11 on one side of an outer end thereof.

The second strap 20 is made of fabric and has a second coupling portion 21 on one side of an outer end thereof. The second coupling portion 21 is configured for connecting the first coupling portion 11 of the first strap 10. In this embodiment, one of the first coupling portion 11 and the second coupling portion 21 is an adhesive strap having tiny hooks, and the other of the first coupling portion 11 and the second coupling portion 21 is an adhesive strap having soft loops, but not limited thereto. The first coupling portion 11 and the second coupling portion 21 may be other types of fasteners.

The two first protective sheets 30 are made of a rigid material and connected to the inner ends of the first strap 10 and the second strap 20, respectively. The first protective sheet 30 is non-planar. The middle portion of the first protective sheet 30 protrudes outward to form a first recessed space 30A on the inner surface of the first protective sheet 30. When a user wears the waist protector, through the first recessed space 30A, only two sides of the first protective sheet 30 are in contact with the user's body, and the middle portion of the first protective sheet 30 is not in contact with the user's body. The first protective sheet 30 is formed with a plurality of first ventilation holes 31. The upper and lower ends of the first protective sheet 30 each have a first curved extension section 32 extending outward. The first ventilation holes 31 are in communication with the first recessed space 30A. In this embodiment, the first protective sheet 30 has a pointed, arched cross-section, but is not limited thereto. The rigid material may be metal, plastic, composite materials, and the like, providing good strength and rigidity. The inner end of the first strap 10 passes through the first ventilation holes 31 of one of the first protective sheets 30 and is folded back and fixed by sewing. The inner end of the second strap 10 passes through the first ventilation holes 31 of the other first protective sheet 30 and is folded back and fixed by sewing. The first ventilation holes 31 of the first protective sheet 30 each have a rounded rectangle shape.

The second protective sheet 40 is made of a rigid material and located between the two first protective sheets 30 and connected to each of the two first protective sheets 30 through at least one connecting member 50. The second protective sheet 40 is non-planar. The middle portion of the second protective sheet 40 protrudes outward to form a second recessed space 40A on the inner surface of the second protective sheet 40. When the user wears the waist protector, through the second recessed space 40A, only two sides of the second protective sheet 40 are in contact with the user's body, and the middle portion of the second protective sheet 40 is not in contact with the user's body. The second protective sheet 40 is formed with a plurality of second ventilation holes 41. The upper end of the second protective sheet 40 has a second curved extension section 42 extending outward. The second ventilation holes 41 are in communication with the second recessed space 40A. In this embodiment, the second protective sheet 40 has a pointed, arched cross-section, but is not limited thereto. The rigid material may be metal, plastic, composite materials, and the like, providing good strength and rigidity. The second ventilation holes 41 of the second protective sheet 40 each have a rounded rectangle shape. The connecting member 50 is a tension spring with elasticity, and has two ends respectively hooked on the first protective sheet 30 and the second protective sheet 40, but not limited thereto. The connecting member 50 may be an elastic rope, an elastic band, etc. Each of the two corners of the lower end of the second protective sheet 40 has a curved protective portion 43 extending outward, thereby achieving the effect of protection.

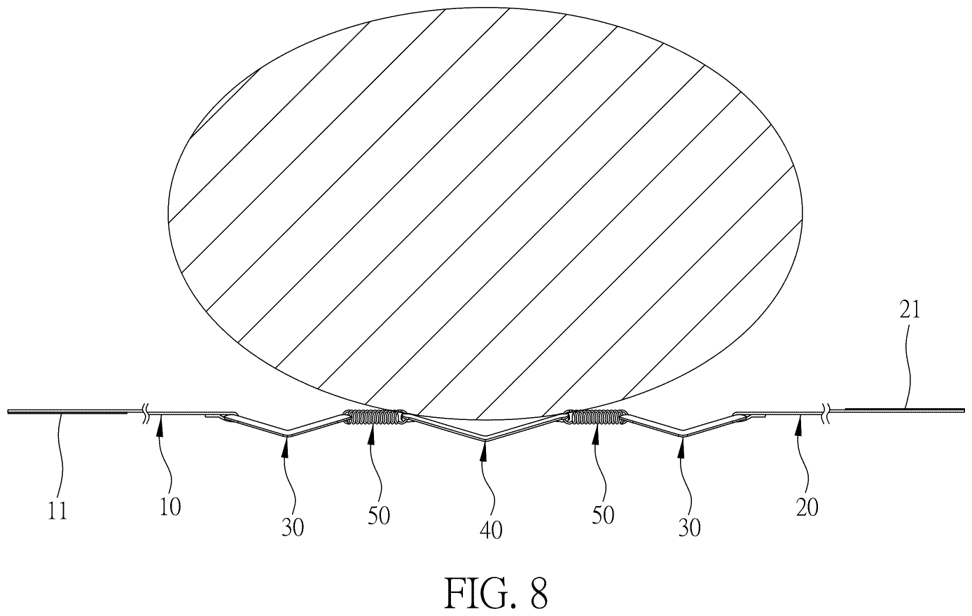

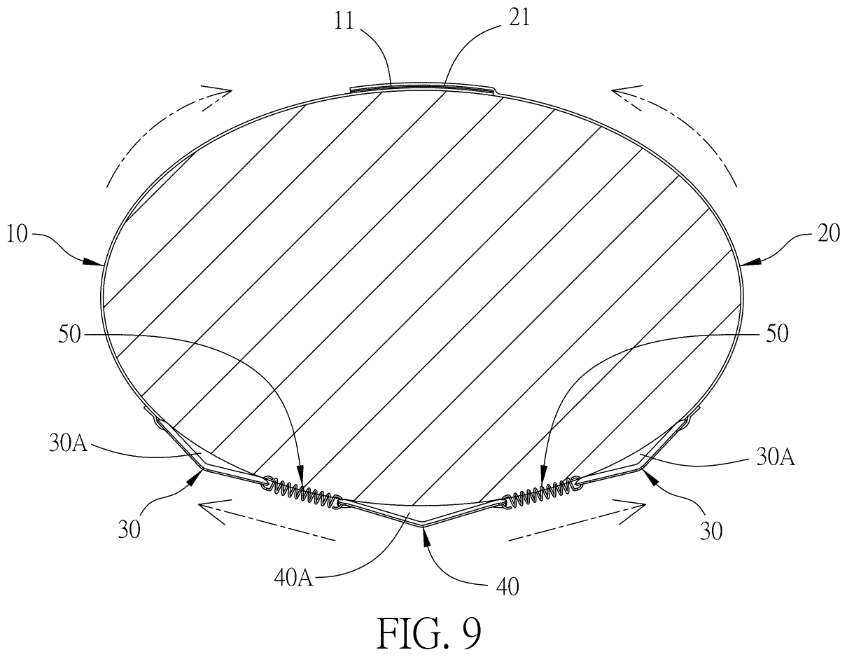

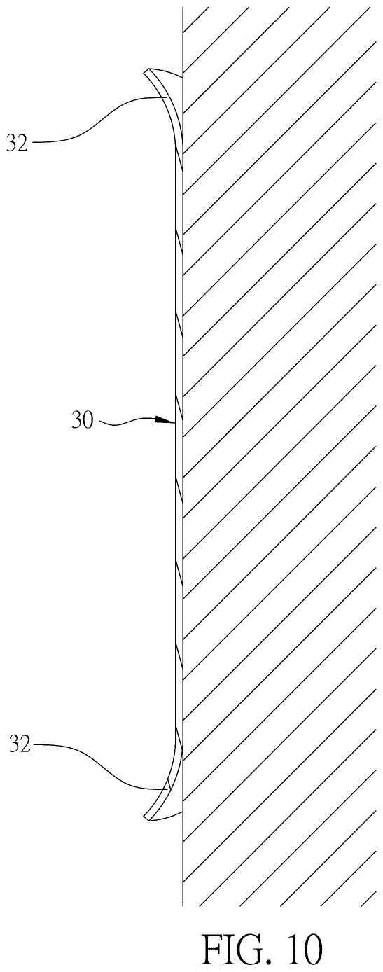

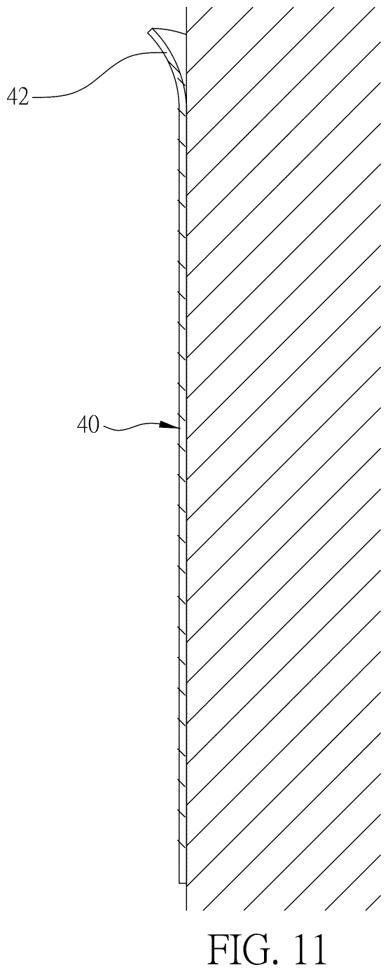

When the user wants to wear the waist protector provided by the present invention on the waist, as shown in FIGS. 8-11, the second protective sheet 40 is aligned with the user′ lumbar, and then the first strap 10 and the second strap 20 surround the waist of the user from back to front so that the two first protective sheets 30 are tightly attached to both sides of the waist of the user. When in use, only two sides of the first protective sheet 30 are in contact with the user's body, and the middle portion of the first protective sheet 30 is not in contact with the user's body through the first recessed space 30A. The second protective sheet 40 is tightly attached to both sides of the user's lumbar. Only two sides of the second protective sheet 40 are in contact with the user's body, and the middle portion of the second protective sheet 40 is not in contact with the user's body and lumbar through the second recessed space 40A. Then, the first coupling portion 11 and the second coupling portion 21 are attached to each other to achieve a tightening effect, so as to provide lumbar support, reduce the burden on the lumbar, reduce the risk of injury, and disperse and reduce the pressure on the lumbar effectively, thereby reducing the lumbar fatigue and promoting recovery from lumbar injury or surgery.

The first curved extension sections 32 on the upper and lower ends of the first protective sheet 30 and the second curved extension section 42 on the upper end of the second protective sheet 40 are configured for expanding the user's range of motion, allowing the user to bend his/her body freely when wearing the waist protector.

With the structure of the above specific embodiment, the following benefits can be obtained:
1. In the present invention, the first protective sheets 30 and the second protective sheet 40 are made of a rigid material. The middle portion of the first protective sheet 30 protrudes outward to form the first recessed space 30A on the inner surface of the first protective sheet 30. The first protective sheet 30 is formed with the plurality of first ventilation holes 31 that are in communication with the first recessed space 30A. The middle portion of the second protective sheet 40 protrudes outward to form the second recessed space 40A on the inner surface of the second protective sheet 40. The second protective sheet 40 is formed with the plurality of second ventilation holes 41 that are in communication with the second recessed space 40A. The middle portion of the first protective sheet 30 is not in contact with the user's body because of the first recessed space 30A, and the middle portion of the second protective sheet 40 is not in contact with the user's body because of the second recessed space 40A. The first recessed space 30A is ventilated with the external air through the first ventilation holes 31, and the second recessed space 40A is ventilated with the external air through the second ventilation holes 41, thereby improving the overall air permeability, effectively reducing the stuffy feeling when the user wears the waist protector, and thus improving the wearing comfort.
2. In the present invention, the middle portion of the second protective sheet 40 protrudes outward to form the second recessed space 40A on the inner surface of the second protective sheet 40. When in use, only two sides of the second protective sheet 40 are in contact with the user's body, and the middle portion of the second protective sheet 40 is not in contact with the user's body and lumbar because of the second recessed space 40A, thereby reducing the pressure on the user's lumbar. This is ergonomically designed to avoid discomfort and inconvenience, thus enhancing practicality.

While the embodiments of the invention have been set forth for the purpose of disclosure, modifications of the disclosed embodiments of the invention as well as other embodiments thereof, may occur to those skilled in the art. The scope of the claims should not be limited by the embodiments set forth in the examples, but should be given the broadest interpretation consistent with the description as a whole.

1. A multi-piece breathable waist protector comprising:
a first strap having a first coupling portion on one side of an outer end thereof;
a second strap having a second coupling portion on one side of an outer end thereof, wherein the second coupling portion is configured to connect to the first coupling portion of the first strap;
two first protective sheets, each made of a rigid material and connected to respective inner ends of the first strap or of the second strap, each first protective sheet being non-planar and each having a middle portion protruding outward to form a first recessed space on an inner surface thereof, the first protective sheets each being formed with a plurality of first ventilation holes, the first ventilation holes being in communication with the respective first recessed space, wherein when a user wears the multi-piece breathable waist protector, each first recessed space is held away by two opposed edges of the respective first protective sheet from contact with the user's body; and
a second protective sheet, made of a rigid material and located between the two first protective sheets and connected to each of the two first protective sheets through connecting members, the second protective sheet being non-planar, the second protective sheet having a middle portion protruding outward to form a second recessed space on an inner surface thereof, the second protective sheet being formed with a plurality of second ventilation holes, the second ventilation holes being in communication with the second recessed space, wherein when the user wears the multi-piece breathable waist protector, the second recessed space is held away by two opposed edges of the second protective sheet from contact with the user's body.

2. The multi-piece breathable waist protector as claimed in claim 1, wherein upper and lower ends of each first protective sheet have a first curved extension section extending outward.

3. The multi-piece breathable waist protector as claimed in claim 1, wherein an upper end of the second protective sheet has a second curved extension section extending outward.

4. The multi-piece breathable waist protector as claimed in claim 1, wherein one of the first coupling portion and the second coupling portion is an adhesive strap having hooks, and the other of the first coupling portion and the second coupling portion is an adhesive strap having loops.

5. The multi-piece breathable waist protector as claimed in claim 1, wherein each first protective sheet has a pointed, arched cross-section.

6. The multi-piece breathable waist protector as claimed in claim 1, wherein the inner end of the first strap passes through the first ventilation holes of one of the first protective sheets and is folded back and fixed by sewing, and the inner end of the second strap passes through the first ventilation holes of the other first protective sheet and is folded back and fixed by sewing.

7. The multi-piece breathable waist protector as claimed in claim 1, wherein the connecting members are each a tension spring with elasticity.

8. The multi-piece breathable waist protector as claimed in claim 1, wherein the second protective sheet has a pointed, arched cross-section.

9. The multi-piece breathable waist protector as claimed in claim 1, wherein two corners of a lower end of the second protective sheet each has a curved protective portion extending outward.
